(19) United States
(12) Patent Application Publication
(10) Pub. No.: US 2024/0398692 A1
(43) Pub. Date: Dec. 5, 2024

(54) MANUFACTURING METHOD OF COFFEE CELL AND ADVENTITIOUS ROOT CULTURE COMPRISING INCREASED BIOACTIVE SUBSTANCES AND USE THEREOF

(71) Applicant: BIO-FD&C, Incheon (KR)

(72) Inventors: Sang Hyun MOH, Incheon (KR); Jeong Hun LEE, Incheon (KR); Hyo Hyun SEO, Incheon (KR); Soo-Yun KIM, Incheon (KR); Kyeong Yeon HEO, Hanam-si (KR); Min Kyu KIM, Suwon-si (KR); Ji Ohn MOH, Seongnam-si (KR)

(73) Assignee: BIO-FD&C, Incheon (KR)

(21) Appl. No.: 18/680,600

(22) Filed: May 31, 2024

(30) Foreign Application Priority Data
Jun. 2, 2023 (KR) 10-2023-0071642

(51) Int. Cl. A61K 8/9789; A61Q 19/08; C12N 5/00; C12N 5/04; C12N 13/00

(52) U.S. Cl. CPC A61K 8/9789; A61Q 19/08; C12N 5/0018; C12N 5/04; C12N 13/00; C12N 2500/34; C12N 2500/38; C12N 2501/30

(57) ABSTRACT

The present disclosure relates to a manufacturing method of a coffee cell and an adventitious root culture including increased bioactive substances and a food composition such as coffee using the same. The Coffea arabica plant cell or adventitious root produced by culturing the Coffea arabica plant cell or coffee adventitious root manufactured by the method of the present disclosure has an increased content of bioactive substances to increase skin regeneration, anti-aging and anti-wrinkle effects, and thus may be effectively used for related industries thereof.

### CROSS-REFERENCE TO RELATED APPLICATIONS

This application is based on and claims priority from Korean Patent Application No. 10-2023-0071642, filed on Jun. 2, 2023, with the Korean Intellectual Property Office, the disclosure of which is incorporated herein in its entirety by reference.

This work was supported by the Technology Innovation Program (Advanced Technology Center Plus (ATC+)) (20017936, Development of growth factors and antibody drugs using plant cell-based platform technology and global sales expansion of fragrance materials) funded By the Ministry of Trade, Industry and Energy (MOTIE, Korea).

### TECHNICAL FIELD

The present disclosure relates to a manufacturing method of a coffee cell and an adventitious root culture including increased bioactive substances and a use thereof.

### BACKGROUND

Human skin becomes thinner (in the case of intrinsic aging) and loses elasticity over time by internally intrinsic aging in which the secretion of various hormones that control metabolism decreases, the function of immune cells and the activity of cells decline, and the biosynthesis of immune proteins and bio-structural proteins necessary for the body decreases, and externally various pollutants and photoaging caused by ultraviolet rays. When exposed to ultraviolet radiation, keratinocytes and melanocytes in the epidermis are damaged by UVB. Skin aging is divided into chronologic aging, which occurs in areas non-exposed to sunlight, and actinic aging, which occurs when degenerative changes occurring in areas exposed to sunlight is combined with chronologic aging. In the chronologic aging, as characteristic clinical skin findings, there are found fine wrinkles, dermal atrophy, a decrease in the subcutaneous fat layer, etc. During the photoaging, coarse wrinkling and furrowing occur and abnormal elastotic materials accumulate, so that the skin becomes leathery and loose. A phenomenon which may be seen in chronic sun-damaged skin is an abnormal deposition of elastic materials in the upper collagen of the dermis (solar elastosis), an increase in proteoglycan, and a significant decrease in collagen as a main protein of the dermis. Collagen serves to provide strength and tension to the skin, and thus protect the skin from external stimuli or forces, and accounts for 90% of the dermis layer, so that the decrease in collagen is closely related to the skin and aging. In cells constituting the skin, it is known that while cell activity decreases and a signaling system becomes incomplete due to intrinsic aging or photoaging, the biosynthesis of matrix metalloproteinases (MMPs), enzymes that decompose skin tissue, increases and collagen biosynthesis decreases to cause wrinkles and loss of elasticity, and melanin production which causes skin darkening increases. Accordingly, a substance capable of thickening the skin by proliferating skin cells or increasing a substrate material constituting the skin, a substance capable of inhibiting the biosynthesis of MMPs, which decompose collagen, a substrate material constituting the skin, a substance capable of inhibiting melanin biosynthesis, and the like are found to relieve skin symptoms such as wrinkles, loss of elasticity, and darkening of the skin.

Mass-production of secondary metabolites through plant cell culture has difficulty due to instability of cultured cell lines, low productivity, slow growth, and problems with mass cultivation. To overcome such low productivity, in the related art, there was used methods of increasing the productivity of secondary metabolites, including 1) manipulation of nutriments in a culture medium, such as addition of sugar, nitrate, phosphate, growth regulators, and precursors, 2) optimization of a culture environment, such as culture temperature, illumination, and medium acidity, 3) treatment with inducers to increase productivity, 4) cell membrane permeabilization and binary culture for effective recovery of secondary metabolites, and 5) genetic modification and introduction of foreign genes involved in the biosynthesis of secondary metabolites. However, these methods had a problem that the methods may be used only for specific cells or specific metabolites and may not be generally applied to the majority of cell cultures and secondary metabolites.

Meanwhile, methods for culturing cells include a batch culture method in which a culture medium and cells are added once at the beginning and no nutrients are further supplied or removed, and a continuous culture method in which cells are maintained to be continuously cultured without depleting nutrients by adding a culture medium in an incubator, introducing a new culture medium from the outside at a constant rate, and simultaneously draining the old culture medium containing culture products out at the same rate. Among these, the batch culture method has difficulty in mass-production, and thus a perfusion culture method, which maintains cells in a culture container and continuously recovers the medium containing products while supplying a new medium, is attracting attention. In this case, there is disclosed a method for separating cells from a medium using ultrasound, which is a method for culturing plant cells at a high concentration by separating cells from a medium using an ultrasonic cell separator operating by a method of irradiating with ultrasound, stopping the ultrasound irradiation when the cells start to flow out of the cell separator, returning the cells to the inside of a bioreactor by applying back pressure using a pump, and then irradiating with the ultrasound again. However, in this case, the ultrasound is used as a means for separating the cells from the medium, but there was a problem that the ultrasound did not significantly contribute to increasing the content of secondary metabolites in the cells. In addition, a problem has been pointed out due to a lot of necrosis of plant cells caused by ultrasound.

Meanwhile, a component of coffee that gives its unique flavor is caffeine, which is vegetable alkaloid with Chemical Formula of C₈H₁₀N₄O₂. The caffeine is a colorless, odorless, bitter-tasting, needle crystal. The coffee has various tastes, including bitter, sweet, and astringent. It is known that the bitter taste of coffee comes from caffeine, the astringent taste comes from tannin, the sour taste comes from fatty acids, and the sweet taste comes from sugar.

Caffeine, which is contained in large quantities in coffee, has beneficial effects such as stimulating the central nervous system to make the mind clearer, promoting a heart function, serving as a diuretic system to increase the amount of urine, and stimulating the stomach to promote gastric acid secretion. However, excessive caffeine intake may cause caffeine intoxication and gastrointestinal disorder, and also cause irregular heartbeats or headaches, and since caffeine passes through the placenta intactly, pregnant women should be careful because if they drink too much, the caffeine may affect the fetus and even cause miscarriage. In addition, for women who have gone through menopause after middle age, caffeine intake may decrease bone density to have the risk of fractures, and thus health authorities such as the Ministry of Food and Drug Safety have recommended limiting daily caffeine intake to 400 mg or less.

Accordingly, in a Coffea arabica plant cell or adventitious root cultured in a medium composition according to the present disclosure, the content of bioactive substances increased and anti-aging and anti-wrinkle effects were confirmed, better efficacy during radio frequency (RF) treatment was confirmed, and thus the present disclosure was completed.

### SUMMARY

The present disclosure has been made in an effort to provide a manufacturing method of a Coffea arabica plant cell or adventitious root culture or an extract thereof having an increased content of bioactive substances, including the following steps:
(a) isolating leaf or stem tissue of a coffee plant and culturing a plant cell or adventitious root; and
(b) radio-frequency treating the plant cell or adventitious root in step (a).

The present disclosure has also been made in an effort to provide a functional food composition including a Coffea arabica plant cell, a coffee adventitious root culture or an extract thereof having an increased content of bioactive substances as an active ingredient according to the manufacturing method.

The present disclosure has also been made in an effort to provide an anti-aging or anti-wrinkle cosmetic composition including a Coffea arabica plant cell, a coffee adventitious root culture or an extract thereof having an increased content of bioactive substances as an active ingredient according to the manufacturing method.

The present disclosure has also been made in an effort to provide an anti-aging or anti-wrinkle pharmaceutical composition including a Coffea arabica plant cell, a coffee adventitious root culture or an extract thereof having an increased content of bioactive substances as an active ingredient according to the manufacturing method.

The present disclosure has also been made in an effort to provide a method for increasing carotenoid, flavonoid and phenol in a Coffea arabica plant cell or an adventitious root culture including the following steps:
(a) isolating leaf or stem tissue of a coffee plant and culturing a plant cell or adventitious root; and
(b) radio-frequency treating the plant cell or adventitious root in step (a).

An exemplary embodiment of the present disclosure provides a manufacturing method of a Coffea arabica plant cell or adventitious root culture or an extract thereof having an increased content of bioactive substances, including the following steps:
(a) isolating leaf or stem tissue of a coffee plant and culturing a plant cell or adventitious root; and
(b) radio-frequency treating the plant cell or adventitious root in step (a).

Further, another exemplary embodiment of the present disclosure provides a functional food composition including a Coffea arabica plant cell, a coffee adventitious root culture or an extract thereof having an increased content of bioactive substances as an active ingredient according to the manufacturing method.

Further, yet another exemplary embodiment of the present disclosure provides an anti-aging or anti-wrinkle cosmetic composition including a Coffea arabica plant cell, a coffee adventitious root culture or an extract thereof having an increased content of bioactive substances as an active ingredient according to the manufacturing method.

Further, yet another exemplary embodiment of the present disclosure provides an anti-aging or anti-wrinkle pharmaceutical composition including a Coffea arabica plant cell, a coffee adventitious root culture or an extract thereof having an increased content of bioactive substances as an active ingredient according to the manufacturing method.

Further, yet another exemplary embodiment of the present disclosure provides a method for increasing carotenoid, flavonoid and phenol in a Coffea arabica plant cell or an adventitious root culture including the following steps:
(a) isolating leaf or stem tissue of a coffee plant and culturing a plant cell or adventitious root; and
(b) radio-frequency treating the plant cell or adventitious root in step (a).

According to the exemplary embodiments of the present disclosure, the present disclosure relates to a manufacturing method of a coffee cell and an adventitious root culture including increased bioactive substances and a food composition such as coffee using the same. The Coffea arabica plant cell or adventitious root produced by culturing the Coffea arabica plant cell or coffee adventitious root manufactured by the method of the present disclosure has an increased content of bioactive substances to increase skin regeneration, anti-aging and anti-wrinkle effects, and thus may be effectively used for related industries thereof.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a diagram illustrating a coffee cell production process of the present disclosure.

FIG. 2 is a diagram illustrating a coffee adventitious root culture process of the present disclosure.

### DETAILED DESCRIPTION

Hereinafter, exemplary embodiments of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used in the present disclosure are terminologies used to properly express preferred exemplary embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

All technical terms used in the present disclosure, unless otherwise defined, have the meaning as commonly understood by those skilled in the related art of the present disclosure. In addition, although preferred methods and samples are described herein, similar or equivalent methods and samples thereto are also included in the scope of the present disclosure. The contents of all publications disclosed as references in this specification are incorporated in the present disclosure.

Hereinafter, the present disclosure will be described in detail.

The present disclosure provides a manufacturing method of a Coffea arabica plant cell or adventitious root culture or an extract thereof having an increased content of bioactive substances, including the following steps:
(a) isolating leaf or stem tissue of a coffee plant and culturing a plant cell or adventitious root; and
(b) radio-frequency treating the plant cell or adventitious root in step (a).

In the present disclosure, the production process of the coffee cell may be as follows.

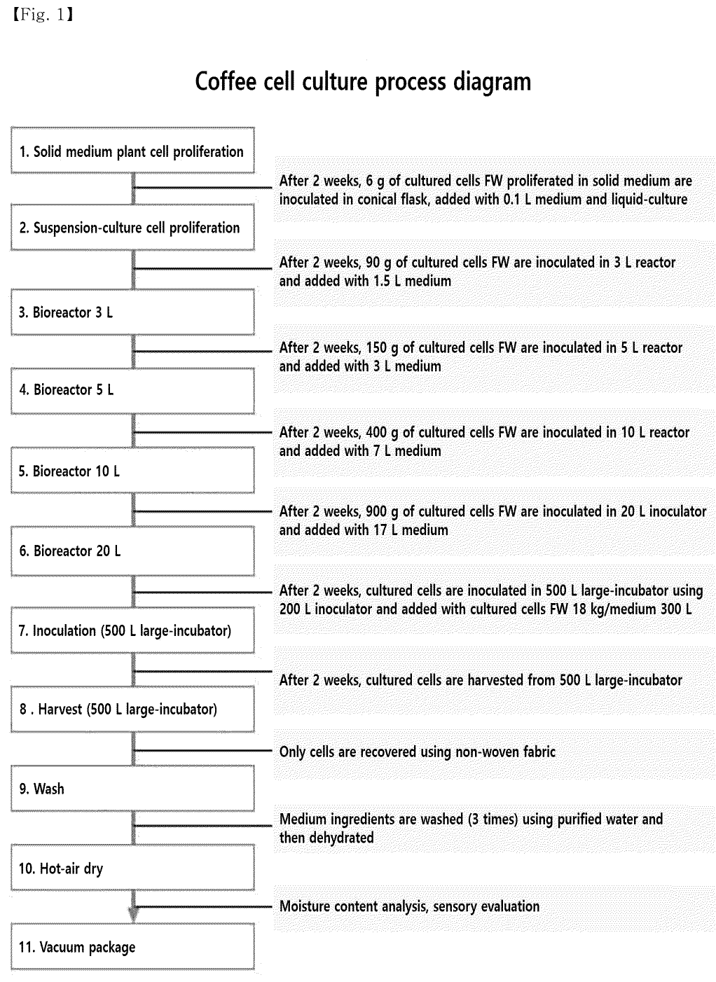

First, 1) plant cells may be proliferated on a solid medium for 1 to 3 weeks, preferably 2 weeks, and then the cells may be proliferated by liquid-culturing the plant cells for 1 to 3 weeks, preferably 2 weeks. Thereafter, 2) the cultured cells may be inoculated into a bioreactor for 1 to 3 weeks, preferably 2 weeks, and then the cells may be further cultured by adding the medium. 3) Thereafter, the process 2) may be repeated 1 to 5 times, and in the case of the repetition, the cells may be cultured while increasing the size of the bioreactor and increasing the amounts of cells and medium. A more specific cell culture process diagram may refer to FIG. 1.

In the present disclosure, a culture process of coffee adventitious root may be as follows.

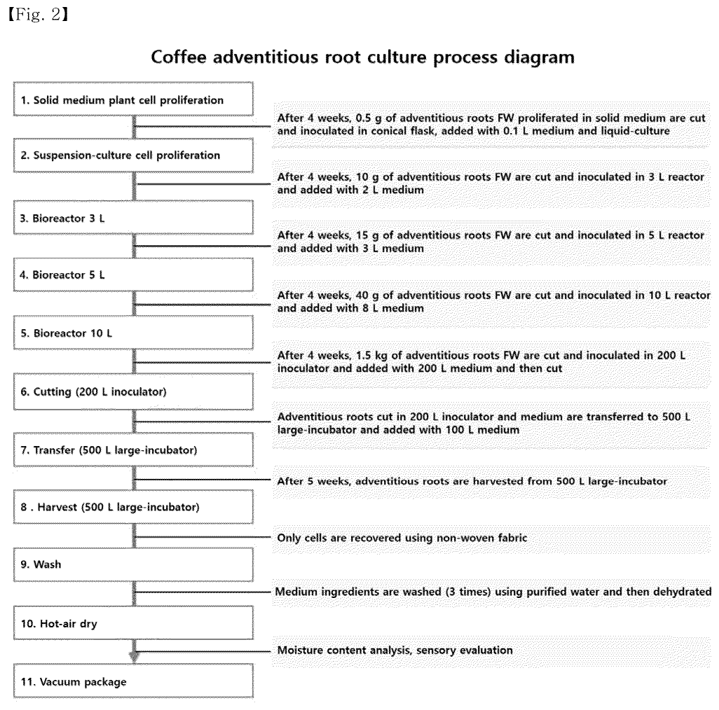

First, the plant cells may be proliferated on a solid medium, and the plant cells may be cultured for 1 to 10 weeks, preferably 5 weeks, and then liquid-cultured for 1 to 10 weeks. Thereafter, the adventitious root may be cut and re-cultured. During the re-culturing, the adventitious root may be re-cultured while increasing the size of the reactor and increasing the amount of adventitious root and the amount of medium. The re-culturing in the bioreactor may be repeated 1 to 5 times, and finally, the cultured adventitious root may be cut and used. A more detailed process may refer to FIG. 2.

In the present disclosure, Coffea arabica plant cells or adventitious roots manufactured by the production process or culture process may be used.

In an example, the step (a) may be
(i) a step of obtaining a plant cell culture by isolating leaf or stem tissue of the coffee plant and culturing the isolated tissue in a medium containing sucrose, myo-inositol, thiamine Hcl, nicotinic acid, and pyridoxine HCl, or
(ii) a step of obtaining an adventitious root culture by isolating the leaf or stem tissue of the coffee plant and culturing the isolated tissue in a medium containing indole-3-butyric acid (IBA), but is not limited thereto.

As an example of the present disclosure, a medium for culturing the coffee cell may include 10 to 50, preferably 30 parts by weight of sucrose, 0.001 to 0.01, preferably 0.005 parts by weight of myo-inositol, 0.001 to 0.01, preferably 0.005 parts by weight of thiamine Hcl, 0.001 to 0.01, preferably 0.005 parts by weight of nicotinic acid, 0.0001 to 0.001, preferably 0.0005 parts by weight of pyridoxine HCl, and 1 to 5, preferably 2.3 parts by weight of Gelrite.

As an example of the present disclosure, a medium for culturing the adventitious root may include 10 to 50, preferably 30 parts by weight of sucrose, 0.0001 to 0.001, preferably 0.0005 parts by weight of indole-3-butyric acid (IBA), and 1 to 5, preferably 2.3 parts by weight of Gelrite.

In an example, the method further includes a step of radio-frequency treating the plant cell or adventitious root cultured in step (a) 3 to 5 times at intervals of 10 to 30 minutes at 30 KHz to 30 MHz, but is not limited thereto.

In an example, step (b) includes a step of preparing a composition by drying and powdering the Coffea arabica plant cell culture or adventitious root culture obtained in step (a) and mixing the culture with purified water, or preparing a composition by powdering and mixing the culture with purified water and then extracting the mixture with an organic solvent, ultrasound or hot water.

Any pharmaceutically acceptable organic solvent may be used as a suitable solvent for extracting the composition of the present disclosure, and water or an organic solvent may be used. For example, as the extraction solvent, various solvents, such as purified water, anhydrous or hydrous lower alcohols having 1 to 4 carbon atoms, including methanol, ethanol, propanol, isopropanol, and butanol, propylene glycol, butylene glycol, glycerin, acetone, ethyl acetate, butyl acetate, chloroform, diethyl ether, dichloromethane, hexane, ether, benzene, methylene chloride, and cyclohexane, may be used alone or in combination.

As the extraction method, any one of methods such as hot water extraction, chilling extraction, reflux cooling extraction, solvent extraction, steam distillation, ultrasonic extraction, elution, and compression may be selected and used. In addition, a desired extract may also be additionally subjected to a conventional fractionation process, and may also be purified using a conventional purification method. There is no limitation to a manufacturing method of the extract of the present disclosure, and any known manufacturing method may be used.

In an example, the bioactive substances are carotenoid, flavonoid, and phenol, but are not limited thereto.

The present disclosure provides a functional food composition including a Coffea arabica plant cell, a coffee adventitious root culture or an extract thereof having an increased content of bioactive substances as an active ingredient according to the manufacturing method.

In an example of the present disclosure, the functional food is in the form of beverage, powder, granule, tablet, soft capsule, hard capsule, liquid, pill, jelly, gummy, candy, cookie, biscuit or balanced nutritional food, and preferably a beverage, and may also be coffee.

In an example, the bioactive substances are carotenoid, flavonoid, and phenol, but are not limited thereto.

In an example, the composition increases an anti-wrinkle effect by increasing the biosynthesis amount of procollagen, but is not limited thereto.

In addition to containing the extract as the active ingredient, the food composition of the present disclosure may contain various flavoring agents or natural carbohydrates as additional ingredients, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The foods capable of adding the composition of the present disclosure include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain pulps for preparing natural fruit juice, fruit juice beverages, and vegetable beverages.

The functional food composition of the present disclosure may be prepared and processed in the form of tablets, capsules, powders, granules, liquids, pills, etc. In the present disclosure, the ‘health functional food composition’ refers to foods prepared and processed by using raw materials or ingredients with functionality, which are useful for the human body according to the Art on Health Functional Foods No. 6727, and means foods taken for adjusting nutrients for the structures and functions of the human body or obtaining a useful effect on health applications such as physiological actions. The health functional food of the present disclosure may include conventional food additives, and the suitability as the food additives is determined by the specifications and standards for the corresponding item in accordance with the general rules of the Food Additives Codex, general test methods, etc., that are approved by the Food and Drug Administration, unless otherwise specified. The items disclosed in the ‘Food Additives Codex’ may include, for example, chemical composites such as ketones, glycine, calcium citrate, nicotinic acid, cinnamic acid, etc.; natural additives such as persimmon color, licorice extract, crystal cellulose, Kaoliang color, guar gum, etc.; mixed formulations such as sodium L-glutamic acid formulations, noodle additive alkali agents, preservative formulations, tar color formulations, etc. For example, the health functional food in the form of tablets may be formed by granulating a mixture obtained by mixing the active ingredient of the present disclosure with an excipient, a binder, a disintegrant, and other additives, and then compression-molding the mixture by adding a slip modifier and the like, or directly compressing the mixture. In addition, the health functional food in the form of tablets may also contain a flavors enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture obtained by mixing the active ingredient of the present disclosure with an excipient, a binder, a disintegrant, etc. by conventional known methods, and may also be coated with white sugar or other coating agents or surface-coated with materials such as starch and talc, if necessary. The health functional food in the form of granules may be prepared by granulizing a mixture obtained by mixing the active ingredient of the present disclosure with an excipient, a binder, a disintegrant, etc. by conventional known methods and may contain a flavoring agent, a flavors enhancer, etc., if necessary.

The present disclosure provides an anti-aging or anti-wrinkle cosmetic composition including a Coffea arabica plant cell, a coffee adventitious root culture or an extract thereof having an increased content of bioactive substances as an active ingredient according to the manufacturing method.

In an example of the present disclosure, the composition regenerates the skin, but is not limited thereto.

In an embodiment of the present disclosure, the composition increases the anti-aging effect by increasing TERT expression, but is not limited thereto.

In an embodiment of the present disclosure, the composition increases an anti-wrinkle effect by increasing the biosynthesis amount of procollagen, but is not limited thereto.

The cosmetics of the present disclosure may include a composition selected from the group consisting of water-soluble vitamins, oil-soluble vitamins, high-molecular peptides, high-molecular polysaccharides, sphingolipids, and seaweed extracts.

The water-soluble vitamins may include any vitamin that may be blended with cosmetics, but preferably include vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, etc., and salts (thiamine hydrochloride, sodium ascorbate, etc.) and derivatives (sodium ascorbic acid-2-phosphate, magnesium ascorbic acid-2-phosphate, etc.) thereof are also included in the water-soluble vitamins which may be used in the present disclosure. The water-soluble vitamins may be obtained by conventional methods, such as microbial transformation, purification from a culture solution of a microorganism, enzymatic or chemical synthesis.

The oil-soluble vitamins may include any vitamin that may be blended with cosmetics, but preferably include vitamin A, carotene, vitamin D2, vitamin D3, vitamin E (d1-α tocopherol, d-α tocopherol, d-α tocopherol), etc., and derivatives thereof (ascorbyl palmitate, ascorbyl stearate, ascorbyl dipalmitate, DL-α tocopherol acetate, DL-α tocopherol nicotinic acid vitamin E, DL-pantothenyl alcohol, D-pantothenyl alcohol, pantothenyl ethyl ether, etc.), etc. are also included in the oil-soluble vitamins used in the present disclosure. The oil-soluble vitamins may be obtained by conventional methods, such as microbial transformation, purification from a culture solution of a microorganism, enzymatic or chemical synthesis.

The high-molecular peptides may include any high-molecular peptide that may be blended with cosmetics, but preferably include collagen, hydrolyzed collagen, gelatin, elastin, hydrolyzed elastin, keratin, etc. The high-molecular peptides may be purified and obtained by conventional methods such as purification from microbial cultures, enzymatic methods, or chemical synthesis, or may be usually purified and used from natural products such as the dermis of pig or cow, fibroin of silkworms, or the like.

The high-molecular polysaccharides may be any high-molecular polysaccharide that may be blended with cosmetics, but preferably include hydroxyethyl cellulose, xanthan gum, sodium hyaluronate, chondroitin sulfate or salts thereof (sodium salt, etc.). For example, chondroitin sulfate or salts thereof, etc. may generally be purified and used from mammals or fish.

The sphingolipids may be any sphingolipid that may be blended with cosmetics, but preferably include ceramide, phytosphingosine, glycosphingolipid, etc. The sphingolipids may generally be purified from mammals, fish, shellfish, yeast, or plants by conventional methods or obtained by chemical synthesis.

In addition to the essential ingredients, the cosmetics of the present disclosure may contain other ingredients that may be generally blended with the cosmetics as needed. Other mixed ingredients that may be added herein may include fat ingredients, moisturizers, emollients, surfactants, organic and inorganic pigments, organic powders, UV absorbers, antiseptics, bactericides, antioxidants, plant extracts, pH regulating agents, alcohols, pigments, aromatics, blood flow stimulants, cooling agents, antiperspirants, purified water, etc. The fat ingredient may include ester-based fat, hydrocarbon-based fat, silicone-based fat, fluorine-based fat, animal fat, vegetable fat, etc.

The ester-based fat may include glyceryl tri2-ethylhexanoate, cetyl 2-ethylhexanoate, isopropyl myristate, butyl myristate, isopropyl palmitate, ethyl stearate, octyl palmitate, isocetyl isostearate, butyl stearate, ethyl linoleate, isopropyl linoleate, ethyl oleate, isocetyl myristate, isostearyl myristate, isostearyl palmitate, octyldodecyl myristate, isocetyl isostearate, diethyl sebacate, diisopropyl adipate, isoalkyl neopentanoate, glyceryl tricaprylate/tricaprate, trimethylolpropane tri2-ethylhexanoate, trimethylolpropane triisostearate, pentaelislitol tetra2-ethylhexanoate, cetyl caprylate, decyl laurate, hexyl laurate, decyl myristate, myristyl myristate, cetyl myristate, stearyl stearate, decyl oleate, cetyl ricinooleate, isostearyl laurate, isotridecyl myristate, isocetyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol dioleate, propylene glycol dicaprate, propylene glycol dicaprylate/dicaprate, propylene glycol dicaprylate, neopentyl glycol dicaprate, neopentyl glycol dioctanate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerol oleate ester, polyglycerol isostearate ester, triisocetyl citrate, triisoalkyl citrate, triisooctyl citrate, lauryl lactate, myristyl lactate, cetyl lactate, octyldecyl lactate, triethyl citrate, acetyl triethyl citrate, acetyl tributyl citrate, trioctyl citrate, diisostearyl malate, 2-ethylhexyl hydroxystearate, di2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stealoyl hydroxystearate, stearyl 12-stealoyl hydroxystearate, isostearyl 12-stealoyl hydroxystearate, etc.

The hydrocarbon-based fat may include squalene, liquid paraffin, α-olefin oligomer, isoparaffin, ceresin, paraffin, liquid isoparaffin, polybutene, microcrystalline wax, Vaseline, etc.

The silicone-based fat may include polymethyl silicone, methylphenyl silicone, methylcyclopolysiloxane, octamethylpolysiloxane, decamethylpolysiloxane, dodecamethylcyclosiloxane, a dimethylsiloxane/methylcetyloxysiloxane copolymer, a dimethylsiloxane/methylstealoxysiloxane copolymer, alkyl-modified silicone oil, amino-modified silicone oil, etc.

The fluorine-based fat may include perfluoropolyether, etc.

The animal or vegetable fat may include animal or vegetable fat, such as avocado oil, almond oil, olive oil, sesame oil, rice bran oil, safflower seed oil, soybean oil, corn oil, rapeseed oil, apricot seed oil, palm kernel oil, palm oil, castor oil, sunflower oil, coffee seed oil, cottonseed oil, coconut oil, candlenut oil, wheat germ oil, rice germ oil, shea butter, evening primrose oil, macadamia nut oil, meadowfoam oil, egg yolk oil, beef tallow, horse oil, mink oil, orange roughy oil, jojoba oil, candelilla wax, carnauba wax, liquid lanolin, hydrogenated castor oil, etc.

The moisturizer may include a water-soluble low-molecular moisturizer, a fat-soluble molecular moisturizer, a water-soluble polymer, a fat-soluble polymer, etc.

The water-soluble low-molecular moisturizer may include serine, glutamine, sorbitol, mannitol, sodium pyrrolidone-carboxylate, glycerin, propylene glycol, 1,3-butylene glycol, ethylene glycol, polyethylene glycol B (degree of polymerization n=2 or more), and polypropylene glycol (degree of polymerization n=2 or more), polyglycerin B (degree of polymerization n=2 or more), lactic acid, lactate, etc.

The fat-soluble low-molecular moisturizer may include cholesterol, cholesterol esters, etc.

The water-soluble polymer may include carboxyvinyl polymer, polyaspartate, tragacanth, xanthan gum, methyl cellulose, hydroxymethyl cellulose, hydroxyethyl cellulose, hydroxypropyl cellulose, carboxymethyl cellulose, water-soluble chitin, chitosan, dextrin, etc.

The fat-soluble polymer may include a polyvinylpyrrolidone/eicosene copolymer, a polyvinylpyrrolidone/hexadecene copolymer, nitrocellulose, dextrin fatty acid ester, polymer silicone, etc. The emollient may include long-chain acyl glutamic acid cholesteryl ester, hydroxystearic acid cholesteryl, 12-hydroxystearic acid, stearic acid, rosin acid, lanolin fatty acid cholesteryl ester, etc.

The surfactant may include a non-ionic surfactant, an anionic surfactant, a cationic surfactant, an amphoteric surfactant, etc.

The non-ionic surfactant may include self-emulsified monostearic acid glycerine, propylene glycol fatty acid ester, glycerine fatty acid ester, polyglycerine fatty acid ester, sorbitan fatty acid ester, polyoxyethylene (POE) sorbitan fatty acid ester, POE sorbite fatty acid ester, POE glycerine fatty acid ester, POE alkylether, POE fatty acid ester, POE hydrogenated caster oil, POE castor oil, a polyoxyethylene (POE)/polyoxypropylene (POP) copolymer, POE/POP alkylether, polyether-modified silicon, lauric acid alkanol amide, alkylamine oxide, hydrogenated soybean phospholipid, etc.

The anionic surfactant may include fatty acid soap, α-acylsulfonate, alkylsulfonate, alkylarylsulfonate, alkylnaphthalenesulfonate, alkylsulfate, POE alkylethersulfate, alkylamidesulfate, alkylphosphate, POE alkylphosphate, alkylamidephosphate, alkyloylalkyltaurate, N-acylamino acid salt, POE alkylethercarboxylate, alkylsulfosuccinate, sodium alkylsulfoacetate, acylated hydrolyzed collagen peptide salt, perfluoroalkyl phosphate ester, etc.

The cationic surfactant may include alkyltrimethyl ammonium chloride, stearyltrimethyl ammonium chloride, stearyltrimethylammonium bromide, cetostearyltrimethyl ammonium chloride, distearyldimethyl ammonium chloride, stearyldimethylbenzyl ammonium chloride, behenyltrimethyl ammonium bromide, benzalkonium chloride, diethylaminoethylamide stearate, dimethylaminopropylamide stearate, quaternary ammonium derivatives of lanolin, etc.

The amphoteric surfactant may include carboxybetaine-type, amide betaine-type, sulfobetaine-type, hydroxyl sulfobetaine-type, amide sulfobetaine-type, phosphobetaine-type, aminocarboxylate-type, imidazoline derivative-type, amideamine-type amphoteric surfactants, etc.

The organic and inorganic pigments may include inorganic pigments such as silicic acid, silica, magnesium silicate, talc, sericite, mica, kaolin, rouge, clay, bentonite, titan-coated mica, bismuth oxychloride, zirconium oxide, magnesium oxide, zinc oxide, titanium oxide, aluminium oxide, calcium sulfate, barium sulfate, magnesium sulfate, calcium carbonate, magnesium carbonate, iron oxide, ultramarine, chromium oxide, chromium hydroxide, calamine, and composites thereof; organic pigments such as polyamide, polyester, polypropylene, polystyrene, polyurethane, vinyl resin, urea resin, phenol resin, fluorine resin, silica resin, acrylic resin, melamine resin, epoxy resin, polycarbonate resin, a divinylbenzene/styrene copolymer, silk powder, cellulose, CI Pigment Yellow, CI Pigment Orange, etc.; and complex pigments of these inorganic pigments and the organic pigments, etc.

The organic powder may include metallic soap such as calcium stearate; metal alkylphosphate such as zinc sodium cetylate, zinc laurylate or calcium laurylate; polyvalent acylamino acid metal salt such as N-lauroyl-β-alanine calcium, N-lauroyl-β-alanine zinc or N-lauroyl glycine calcium; polyvalent amide sulfonic acid metal salt such as N-lauroyl-taurine calcium or N-palmitoyl-taurine calcium; N-acyl basic amino acid such as N-ε-lauroyl-L-lysine, N-ε-palmitoyllysine, N-α-palmitoyl ornithine, N-α-lauroylarginine, or N-α-hydrogenated tallow fatty acid acyl arginine; N-acylpolypeptide such as N-lauroylglycylglycine; α-amino fatty acid such as α-amino caprylic acid, or α-amino lauric acid; polyethylene, polypropylene, nylon, polymethylmethacrylate, polystyrene, divinylbenzene/styrene copolymer, tetrafluoroethylene, etc.

The UV absorber may include para-amino benzoic acid, ethyl-para-amino benzoate, amyl-para-amino benzoate, octyl-para-amino benzoate, salicylic acid ethylene glycol, phenyl salicylate, octyl salicylate, benzyl salicylate, butylphenyl salicylate, homomentyl salicylate, benzyl cinnamate, 2-ethoxyethyl para-methoxy cinnamate, octyl para-methoxy cinnamate, mono-2-ethyl hexane glyceryl di-para-methoxy cinnamate, isopropyl para-methoxy cinnamate, a diisopropyl/diisopropyl cinnamic acid ester mixture, urocanic acid, ethyl urocanate, hydroxy methoxybenzophenone, hydroxyl methoxybenzophenone sulfonic acid and salts thereof, dihydroxy methoxybenzophenone, sodium dihydroxy methoxybenzophenone disulfonate, dihydroxy benzophenone, tetrahydroxy benzophenone, 4-tert-butyl-4′-methoxy dibenzoylmethane, 2,4,6-trianilino-p-(carbo-2′-ethylhexyl-1′-oxy)-1,3,5-triazine, 2-(2-hydroxy-5-methylphenyl)benzotriazole, etc.

The bactericide may include hinokitiol, triclosan, trichlorohydroxydiphenyl ether, chlorhexidine gluconate, phenoxy ethanol, resorcin, isopropylmethylphenol, azulene, salicylic acid, zinc pyrithione, benzalkonium chloride, photosensitizer 301, sodium mononitro guaiacol, undecylenic acid, etc.

The antioxidant may include butylhydroxy anisole, propyl gallate, erythorbic acid, etc.

The pH regulating agent may include citric acid, sodium citrate, malic acid, sodium malate, fumaric acid, sodium fumarate, succinic acid, sodium succinate, sodium hydroxide, disodium hydrogen phosphate, etc.

The alcohol may include a higher alcohol such as cetyl alcohol.

In addition, a blending ingredient which may be added herein is not limited thereto, and any ingredient may also be blended in a range that the objects and effects of the present disclosure are not hindered.

The cosmetics of the present disclosure may be prepared in the form of solutions, emulsions or viscous mixtures.

The ingredients included in the cosmetic composition of the present disclosure may include ingredients which are generally used in the cosmetic composition as active ingredients, and for example, include conventional adjuvants and carriers, such as a stabilizing agent, a solubilizing agent, vitamins, a pigment and an aromatic.

The cosmetic composition of the present disclosure may be prepared in any formulation commonly prepared in the art, and examples thereof may include emulsions, creams, lotions, packs, foundations, lotions, essences, hair cosmetics, etc.

Specifically, the cosmetic composition of the present disclosure includes formulations of skin lotion, skin softener, skin toner, milky lotion, astringent, lotion, moisture lotion, nutritional lotion, massage cream, nutritional cream, moisture cream, hand cream, foundation, essence, nutritional essence, pack, soap, cleansing foam, cleansing lotion, cleansing cream, hair lotion, hair tonic, hair essence, hair shampoo, hair rinse, hair treatment, body lotion and body cleanser.

When the formulation of the present disclosure is the paste, cream, or gel, as the carrier ingredient, animal fiber, vegetable fiber, wax, paraffin, starch, tragacanth, cellulose derivatives, polyethylene glycol, silicone, bentonite, silica, talc, zinc oxide or the like may be used.

When the formulation of the present disclosure is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the present disclosure is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or sorbitan fatty acid ester.

When the formulation of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

The present disclosure provides an anti-aging or anti-wrinkle pharmaceutical composition including a Coffea arabica plant cell, a coffee adventitious root culture or an extract thereof having an increased content of bioactive substances as an active ingredient according to the manufacturing method.

As used in the present disclosure, the term “prevention” refers to all actions that suppress the symptoms of a specific disease or delays its progression by administering the composition of the present disclosure.

As used in the present disclosure, the term “treatment” refers to all actions that alleviate or beneficially change the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or incomplete adjuvant to increase the immunity thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, and sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one excipient, for example, starch, calcium carbonate, sucrose, lactose, gelatin, etc. with the active ingredients. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base of the suppository, witepsol, Tween 61, cacao butter, laurinum, glycerogelatin, etc. may be used.

The pharmaceutical composition of the present disclosure may be administered to a subject through various routes. All methods of administration may be expected, and the pharmaceutical composition may be administered, for example, oral, intravenous, intramuscular, subcutaneous, and intraperitoneal injection.

The pharmaceutical composition may be formulated into various oral or parenteral dosage forms.

Formulations for oral administration include, for example, tablets, pills, hard and soft capsules, solutions, suspensions, emulsifiers, syrups, granules, etc., and these formulations may further include diluents (e.g., lactose, dextrose, sucrose, mannitol, sorbitol, cellulose and/or glycine), lubricants (e.g., silica, talc, stearic acid and its magnesium or calcium salts and/or polyethylene glycol), in addition to the active ingredient. In addition, the tablets may contain a binder such as magnesium aluminum silicate, starch paste, gelatin, tragacanth, methylcellulose, sodium carboxymethylcellulose and/or polyvinylpyrrolidine, and in some cases, may contain disintegrants or effervescent mixtures such as starch, agar, alginic acid or its sodium salt and/or an absorbent, a coloring agent, a flavoring agent and a sweetening agent. The formulations may be prepared by conventional mixing, granulating or coating methods.

In addition, a typical formulation for parenteral administration is an injection formulation, and as a solvent for the injection formulation, water, a Ringer's solution, isotonic physiological saline or a suspension may be used. Sterile fixed oils of the injection formulation may be used as a solvent or suspension medium, and any non-irritating fixed oil including mono- and di-glycerides may be used for this purpose. In addition, the injection formulation may use fatty acids such as oleic acid.

The present disclosure provides a method for increasing carotenoid, flavonoid and phenol in a Coffea arabica plant cell or an adventitious root culture including the following steps:
(a) isolating leaf or stem tissue of a coffee plant and culturing a plant cell or adventitious root; and
(b) radio-frequency treating the plant cell or adventitious root in step (a).

In an example of the present disclosure, step (a) includes
(i) a step of obtaining a plant cell culture by isolating leaf or stem tissue of the coffee plant and culturing the isolated tissue in a medium containing sucrose, myo-inositol, thiamine Hcl, nicotinic acid, and pyridoxine HCl, or
(ii) a step of obtaining an adventitious root culture by isolating the leaf or stem tissue of the coffee plant and culturing the isolated tissue in a medium containing indole-3-butyric acid (IBA), but is not limited thereto.

Hereinafter, the present disclosure will be described in more detail with reference to Examples. However, the following Examples are only examples to easily explain the content and scope of the technical idea of the present disclosure, and the technical scope of the present disclosure is not limited or changed thereby. In addition, based on these Examples, it may be easily determined by those skilled in the art that various modifications and changes are possible within the scope of the technical idea of the present disclosure.

### Example 1. Induction of Coffea arabica Plant Cell

Coffea arabica used in the present disclosure was immersed in 70% ethanol for 30 seconds and then washed with sterilized water, disinfected again with 0.5% sodium hypochlorite disinfectant for 20 minutes, rinsed three times with sterilized water, and then wounded once using a sharp knife and dark-cultured in a basic MS medium (Murashige and Skoog 1962, Duchefa CO., Ltd., Cat No. M0221) containing 30 g Sucrose, 1 g Myo-Inositol, 5 mg Thiamine Hcl, 5 mg Nicotinic acid, 0.5 mg Pyridoxine HCl and 2.3 g Gelrite per 1 L under growth conditions of 23±2° C. and 70% humidity to induce an initial coffee cell. During the preparation of the medium, the pH was adjusted to 5.8 using 1N sodium hydroxide (NaOH). The subculture of the induced coffee cell was performed at 2 to 3 week intervals and proliferated (FIG. 1).

### Example 2. Induction of Coffea arabica Plant Cell Adventitious Root

To induce adventitious roots from a coffee plant, the adventitious roots were induced by dark-culturing the coffee plant in a ½ basic MS medium containing 50 g Sucrose, 0.5 mg Indole-3-butyric Acid (IBA), and 2.3 g Gelrite per 1 L under growth conditions of 23±2° C. and 70% humidity for 4 weeks. During the preparation of the medium, the pH was adjusted to 5.8 using 1N sodium hydroxide (NaOH). The subculture of the induced coffee adventitious root was performed at 3 to 5 week intervals (FIG. 2).

### Example 3. Mass Production of Coffea arabica Plant Cells and Adventitious Roots According to the Present Disclosure

To mass-produce Coffea arabica plant cells and adventitious roots aseptically derived and cultured from a coffee plant, in the Coffea arabica plant cells of Example 1, the Coffea arabica plant cells derived and proliferated in the basic MS medium containing 30 g Sucrose, 1 g Myo-Inositol, 5 mg Thiamine Hcl, 5 mg Nicotinic acid, 0.5 mg Pyridoxine HCl and 2.3 g Gelrite per 1 L were inoculated and suspension-cultured.

In addition, in the case of the coffee adventitious roots in Example 2, the tips of adventitious roots induced and proliferated in a ½ basic MS medium containing 50 g Sucrose, 0.5 mg Indole-3-butyric Acid (IBA), and 2.3 g Gelrite per 1 L were cut to about 1 cm and inoculated and suspension-cultured. In a culture chamber at 23±2° C., under humidity of 70%, the adventitious roots were cultured in a 20 L balloon-type bioreactor (Samsung Science Co., Ltd.) with an air supply of approximately 0.1 vvm for 4 to 5 weeks.

The Coffea arabica plant cells and adventitious roots cultured in the 20 L balloon-type bioreactor were cultured and mass-produced at 4 to 5 week intervals in 100 L, 200 L, 500 L, and 1000 L pilot scale large-incubator.

When the mass-culture was completed, the culture solution was removed and washed several times with sterilized water, and the Coffea arabica plant cells and the adventitious roots were then freeze-dried or hot-air dried, and stored in an airtight package in a powder form.

### Example 4. Increased Content of Bioactive Substances (Carotenoid, Flavonoid, Phenol) in Coffea arabica Plant Cells and Adventitious Roots Through Radio-Frequency Treatment

The Coffea arabica plant cells and the adventitious root culture obtained in Example 3 above were treated with an attractant as follows.

In the case of the Coffea arabica plant cells, after culturing under the same culture conditions, radio-frequency (RF) treatment was performed in an incubator equipped with a RF waveform generator. It was confirmed that the content of bioactive substances was increased by RF treatment of 30 KHz to 30 MHz, and the content of bioactive substances was highest by RF treatment of 100 to 700 KHz (Table 1).

Specifically, the coffee cells and adventitious roots were suspension-cultured in 100 L and 200 L pilot scales, and cultured for 4 weeks in a 500 L incubator to obtain sufficient biomass, and then treated with a radio frequency (360 kHz) once or twice a day at 20-minute intervals. The culture solutions of the treated Coffea arabica plant cells and adventitious roots were removed, washed at least three times using sterilized water and then freeze-dried.

The contents of carotenoid, flavonoid and phenol, which were secondary metabolites of the Coffea arabica plant cells and coffee adventitious roots according to the present disclosure, were measured by the following method.

4-1. Measurement of Total Carotenoid Content in Coffea arabica Plant Cells and Adventitious Roots Using RF Treatment

A total carotenoid content in Coffea arabica plant cells and adventitious roots was measured by RF treatment of the present disclosure according to an AOAC method.

The freeze-dried Coffea arabica plant cell and adventitious root samples were extracted by adding a mixture of acetone:hexane (3:7, v/v) and then filtered, and the resulting extract was washed with distilled water, a chromatographic column was prepared using a mixture of activated magnesia:diatomaceous earth (1:9, v/v), and the absorbance at 436 nm was measured using a mixture of acetone:hexane (1:9, v/v) while the extract was injected and suctioned.

4-2. Measurement of Total Flavonoid Content in Coffea arabica Plant Cells and Adventitious Roots Using RF Treatment

The flavonoid content of Coffea arabica plant cells and adventitious roots by RF treatment of the present disclosure was measured according to a method of Sakanaka et al. (2005) based on a Colorimetric method.

1.25 mL of distilled water was added to 0.25 mL of the Coffea arabica plant cell and adventitious root culture extract and a standard material solution, and then added with 0.075 mL of a 5% (w/v) NaNO₂ solution. After 6 minutes, the mixture was added with 0.15 mL of a 10% (w/v) AlCl₃ solution, left for 5 minutes, and then added with 0.5 mL of a 1N NaOH solution. Distilled water was added so that the final volume of the mixture became 2.5 ml, and the absorbance was measured at a wavelength of 510 nm.

A (+/−) catechin standard solution (Sigma chemical Co., St. Louis, MO, USA) was used as a standard material, and the total flavonoid content was measured as mg·g⁻¹·DW.

4-3. Measurement of Total Phenol Content in Coffea arabica Plant Cells and Adventitious Roots Using RF Treatment

The total phenol content of Coffea arabica plant cells and adventitious roots through the RF treatment of the present disclosure was measured using a method of Ali, et al. (2006a) based on a Foline-Ciocalteu method (Folin and Ciocalteu, 1927) in which a Foline-Ciocalteu reagent was reduced by the total phenol substances of the culture extract of Coffea arabica plant cells and adventitious roots to be developed to molybdenum blue color.

0.05 mL of the extract and a standard material solution were added with 2.55 mL of distilled water, and then added with 0.1 mL of a 2N Folin-Ciocalteu reagent solution (10 time dilution; Sigma chemical Co., St. Louis, MO, USA). After 6 minutes, the mixture was added with 0.5 ml of a 20% (w/v) Na₂CO₃ solution, and then left in the dark for 30 minutes, and then the absorbance was measured at a wavelength of 760 nm.

Garlic acid was used as a standard material, and the total phenol content was measured as mg·g⁻¹·DW.

TABLE 1

| | | | | Phenol |
|---|---|---|---|---|
| | | Carotenoid | Flavonoid | compound |
| | | content | content | content |
| Control | — | 0.0 | 0.0 | 0.0 |
| Coffee plant | RF non- | 15.5 | 23.1 | 37.4 |
| cell | treatment | | | |
| | RF treatment | 90.5 | 150.3 | 260.4 |
| Coffee | RF non- | 25.5 | 34.8 | 40.7 |
| adventitious | treatment | | | |
| root | RF treatment | 80.4 | 140.6 | 200.1 |

As shown in Table 1 above, as a result of measuring carotenoid, flavonoid, and phenol contents of Coffea arabica plant cells and coffee adventitious roots before and after RF treatment, it was confirmed that the content of bioactive substances increased through RF treatment and the antioxidant efficacy increased.

### Example 5. Confirmation of Skin Regeneration Efficacy Through Increased Proliferation and Mobility of Coffea arabica Plant Cells and Adventitious Root Cells Through RF Treatment

In order to confirm a skin regeneration effect through increased proliferation and mobility of Coffea arabica plant cells and adventitious root cells through RF treatment, Wound healing assay was performed.

A culture insert for wound healing assay was added to each well of a 24-well plate, and HaCaT cells were seeded into the insert at <90% confluence and then cultured for 24 hours. Thereafter, the insert was removed, and the cell pattern was obtained through a microscope photograph at 0 time zone, and a control group added with purified water and a composition as a test substance were treated to the cells. After additional culture for 16 hours, the medium was removed, 4% Paraformaldehyde (PFA) was added, and reacted at room temperature for 15 minutes. After the fixing process, the cells were washed three times with PBS, and 16 hours later, the cell pattern was measured by micrograph. The rate of increase in healing area was calculated by measuring an area between cells at 0 and 16 time zones using Image J.

TABLE 2

| Sample name | | Healing Area Rate (%) |
|---|---|---|
| Control | — | 15.6 |
| Coffee | RF non-treatment | 20.4 |
| plant cell | RF treatment | 36.7 |
| Coffee | RF non-treatment | 21.7 |
| adventitious root | RF treatment | 38.9 |

As shown in Table 2 above, it was confirmed that the Coffea arabica plant cell and coffee adventitious root compositions of the present disclosure increased a healing area rate, and during RF treatment according to the present disclosure, the healing area rate was further increased to have excellent skin regeneration.

### Example 6. Confirmation of Anti-Aging Efficacy of Coffea arabica Plant Cells and Adventitious Roots Through RF Treatment

To investigate the anti-aging effect of Coffea arabica plant cells and adventitious roots through RF treatment, real-time PCR was performed to determine the mRNA expression level of TERT as an aging-related factor.

HaCaT cells were dispensed into a 96-well plate and then cultured for 24 hours under cell culture conditions. Thereafter, the cells were treated with a control group and a composition as a test material and further cultured for 24 hours. After sample treatment, SuperPrep™ cell lysis & RT Kit for qPCR (TOYOBO, Cat. SCQ-101) was used for RNA isolation and cDNA synthesis. The cells from which the medium was removed were washed once with PBS, added with 50 μL of a cell lysis mixture, reacted for 5 minutes, and then added with 10 μL of a stop solution. 8 μL of the previously extracted lysate was added to 32 μL of a RT reaction mixture, and cDNA was synthesized using PCR under the conditions of 37° C. for 15 minutes, 50° C. for 5 minutes, and 95° C. for 5 minutes. To compare and analyze gene expression, the cDNA synthesized above was used as a template and real-time PCR analysis was performed using Thunderbird™ SYBR qPCR Mix (TOYOBO, Cat. QPS-201). The primers used in the experiment were QuantiTect primer assays (GAPDH; Cat. QT01192646, TERT; Cat. QT02402351) of Qiagen Co., Ltd., and the TERT mRNA expression level of the sample was quantified using GADPH. Real-time qPCR conditions were performed at total 40 cycles of 94° C. for 15 seconds, 60° C. for 30 seconds, and 72° C. for 30 seconds per cycle after reaction at 95° C. for 1 minute, and then confirmed using a Rotor-Gene Q instrument.

TABLE 3

| | | Relative expression |
|---|---|---|
| Sample name | | level of TERT gene |
| Control | — | 1.0 |
| Coffee plant cell | RF non-treatment | 1.5 |
| | RF treatment | 3.1 |
| Coffee adventitious | RF non-treatment | 1.4 |
| root | RF treatment | 3.9 |

As shown in Table 3 above, it was confirmed that the Coffea arabica plant cell and coffee adventitious root compositions of the present disclosure increased the TERT gene expression level, and during RF treatment according to the present disclosure, the TERT gene expression level was further increased to have excellent anti-aging.

### Example 7. Confirmation of Anti-Wrinkle Efficacy of Coffea arabica Plant Cells and Adventitious Roots Through RF Treatment

To investigate the anti-wrinkle efficacy of Coffea arabica plant cells and adventitious roots through RF treatment, PIP assay was performed to confirm whether to increase the amount of procollagen production related to wrinkle improvement.

Detroit cells were dispensed into a 48-well plate and cultured for 24 hours under cell culture conditions. Thereafter, the cells were treated with a control group added with purified water and a composition as a test material were treated, and further cultured for 24 hours. 20 μL of a portion of the diluted supernatant from the culture medium through the process was taken and added into each antibody-coated microtiter plate together with 20 μL of a PIP standard material in a PIP EIA kit (Takara, Cat. MK101), and 100 μL of a peroxidase-labeled antibody solution was also added to each well, and reacted at 37° C. for 3 hours. After removing the medium, the cells were washed four times with 200 μL of PBS, 100 μL of a substrate solution was added to each well and reacted at room temperature for 15 minutes in a light-shielded state. Thereafter, 100 μL of a stop solution (1N H₂SO₄) was added, and the absorbance at 450 nm was measured. The absorbance at 450 nm was measured, and then a PIP standard concentration curve was prepared to calculate the amount of PIP in the sample.

TABLE 4

| | | Procollagen |
|---|---|---|
| | | biosynthesis amount |
| Sample name | | (% of control) |
| Control | — | 100 |
| Coffee plant cell | RF non-treatment | 112 |
| | RF treatment | 145 |
| Coffee adventitious | RF non-treatment | 115 |
| root | RF treatment | 141 |

As shown in Table 4 above, it was confirmed that the Coffea arabica plant cell and coffee adventitious root compositions of the present disclosure increased the procollagen biosynthesis amount, and during RF treatment according to the present disclosure, the collagen biosynthesis enhancing effect was further increased to have an excellent anti-wrinkle effect.

Hereinabove, the present disclosure has been described with reference to preferred examples thereof. It will be understood to those skilled in the art that the present disclosure may be implemented as modified forms without departing from an essential characteristic of the present disclosure. Therefore, the disclosed examples should be considered in an illustrative viewpoint rather than a restrictive viewpoint. The scope of the present disclosure is illustrated by the appended claims rather than by the foregoing description, and all differences within the scope of equivalents thereof should be construed as being included in the present disclosure.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A manufacturing method of a Coffea arabica plant cell or adventitious root culture or an extract thereof having an increased content of bioactive substances, comprising the following steps:
(a) isolating leaf or stem tissue of a coffee plant and culturing a plant cell or adventitious root; and
(b) radio-frequency treating the plant cell or adventitious root in step (a).

2. The method of claim 1, wherein step (a) comprises
(i) a step of obtaining a plant cell culture by isolating leaf or stem tissue of the coffee plant and culturing the isolated tissue in a medium containing sucrose, myo-inositol, thiamine Hcl, nicotinic acid, and pyridoxine HCl, or
(ii) a step of obtaining an adventitious root culture by isolating the leaf or stem tissue of the coffee plant and culturing the isolated tissue in a medium containing indole-3-butyric acid (IBA).

3. The method of claim 1, wherein the radio-frequency treatment in step (b) further comprises radio-frequency treatment 3 to 5 times at 30 KHz to 30 MHz at intervals of 10 to 30 minute.

4. The method of claim 1, wherein the bioactive substances are carotenoid, flavonoid, and phenol.

5. A functional food composition comprising a Coffea arabica plant cell, a coffee adventitious root culture or an extract thereof having an increased content of bioactive substances according to the manufacturing method of claim 1 as an active ingredient.

6. The composition of claim 5, wherein the functional food is in the form of beverage, powder, granule, tablet, soft capsule, hard capsule, liquid, pill, jelly, gummy, candy, cookie, biscuit or balanced nutritional food.

7. The composition of claim 6, wherein the beverage is coffee.

8. The composition of claim 5, wherein the bioactive substances are carotenoid, flavonoid, and phenol.

9. The composition of claim 5, wherein the composition increases an anti-wrinkle effect by increasing the biosynthesis amount of procollagen.

10. A method for providing an anti-aging or anti-wrinkle effect, the method comprising administering to a skin of a subject in need thereof a composition comprising a Coffea arabica plant cell, a coffee adventitious root culture or an extract thereof having an increased content of bioactive substances according to the manufacturing method of claim 1 as an active ingredient.

11. The method of claim 10, wherein the subject requires regenerating the skin.

12. The method of claim 10, wherein the subject requires increasing an anti-aging effect by increasing TERT expression.

13. The method of claim 10, wherein the subject requires increasing an anti-wrinkle effect by increasing the biosynthesis amount of procollagen.

14. (canceled)

15. A method for increasing carotenoid, flavonoid and phenol in a Coffea arabica plant cell or an adventitious root culture comprising the following steps:
(a) isolating leaf or stem tissue of a coffee plant and culturing a plant cell or adventitious root; and
(b) radio-frequency treating the plant cell or adventitious root in step (a).

16. The method of claim 15, wherein step (a) comprises
(i) a step of obtaining a plant cell culture by isolating leaf or stem tissue of the coffee plant and culturing the isolated tissue in a medium containing sucrose, myo-inositol, thiamine HCl, nicotinic acid, and pyridoxine HCl, or
(ii) a step of obtaining an adventitious root culture by isolating the leaf or stem tissue of the coffee plant and culturing the isolated tissue in a medium containing indole-3-butyric acid (IBA).
